(19) United States
(12) Patent Application Publication
(10) Pub. No.: US 2002/0061538 A1
(43) Pub. Date: May 23, 2002

(54) Fluidic arrays

(76) Inventor: Rajan Kumar, Robbinsville, NJ (US)

Correspondence Address: Rajan Kumar, 18 Buford Road, Robbinsville, NJ, 08691, US

(21) Appl. No.: 10/052,452

(22) Filed: Oct. 25, 2001

Related U.S. Application Data
(60) Provisional application No. 60/243,138, filed on Oct. 26, 2000.
(60) Provisional application No. 60/244,134, filed on Oct. 30, 2000.
(60) Provisional application No. 60/251,332, filed on Dec. 6, 2000.
(60) Provisional application No. 60/268,132, filed on Feb. 13, 2001.

(51) Int. Cl. C12Q 1/68; G01N 33/53; B05D 3/00; G01N 33/542; C12M 1/34

(52) U.S. Cl. 435/6; 435/7.9; 435/287.2; 438/1; 427/2.11

(57) ABSTRACT

This invention describes novel architectures for molecular arrays and methods for using the same. Also described are methods to use the invention in conjunction with fluidic devices. The molecular arrays consist of DNA, RNA, proteins or peptides, or any other molecule of interest. The uses of such arrays include genomic and proteomic analysis, diagnostic assays, and drug discovery.

### CROSS-REFERENCE TO RELATED APPLICATIONS

This application claims the benefit under 35 U.S.C. 119(e) from U.S. Provisional Application 60/243,138, filed Oct. 26, 2000, U.S. Provisional Application 60/244,134, filed Oct. 30, 2000, U.S. Provisional Application 60/251,332, filed Dec. 6, 2000, and U.S. Provisional Application 60/268,132, filed Feb. 13, 2001.

### STATEMENT REGARDING FEDERALLY SPONSORED RESEARCH OR DEVELOPMENT

No Government License Rights

### BACKGROUND OF THE INVENTION

The present invention lies in the field of molecular biology and is particularly concerned with the technique of microarrays used with or without the use of fluidic devices for detection of molecules of interest in a sample, determination of composition of a complex mixture of molecules, and comparison of composition of two or more samples of molecules, such molecules including although not exclusively, DNA, RNA and proteins.

Sequencing of a large number of genomes has generated a growing body of DNA sequence information that promises to revolutionize experimental design and data interpretation in pursuit of biological understanding. However, collection of sequence data, by itself, is not sufficient to decipher the roles of genes and gene products in cellular and organismal function. Therefore, there has been a concomitant growth in development of technologies to exploit the massive amount of DNA sequence data.

One of such revolutionary technologies to emerge in the biotechnology area is the microarray technology. Microarrays, consisting of high-density arrangements of oligonucleotides or complementary DNAs (cDNAs) can be used to interrogate complex mixtures of molecules in a parallel and quantitative manner. When a sample analyzed by microarray technology is derived from a population of mRNA of a cell or cell population, the analysis provides information about the genes that are present in that cell or cell population. Similarly, arrays of proteins, peptides and other small molecules are also being fabricated for analysis of samples for protein-protein interactions, protein-DNA interactions, protein function and drug discovery. The applications of microarrays include diagnostic and environmental testing, genomic research at academic institutions, biotechnology and pharmaceutical companies, and drug discovery.

The procedure to use microarrays is described here with reference to use of DNA microarrays. DNA microarrays are used to measure concentrations of nucleic acid populations in a sample by hybridization. Typically, a large number of DNA fragments (called probes) are attached to a solid substrate to create an array. Each probe is attached to a defined place. The nucleic acids in the sample (called targets) are labeled usually with fluorescent dyes, typically fluorescein, Cy3 and/or Cy5. When the array of probes is exposed to the sample, the target nucleic acids in the sample hybridize to specific probes on the array. By shining light of appropriate wavelength, the array is then visualized to determine which probes are hybridized thereby giving an estimate of the nucleic acids present in the sample.

Typically, microarrays are generated on glass substrates, usually 1 mm thick slides, with a size of 1 inch by 3 inches. The microarrays are created by depositing molecules of interest in defined locations on one surface of the glass substrate. One of the limitations of such arrays is that the number of molecular species that can be included on an array is limited by the amount of surface area available. To increase the number of molecular species that can be deposited on an array surface, and therefore, can be used to simultaneously interrogate a sample, the size of the elements has to be reduced. Such reduction in the size of individual elements has an effect of reducing the sensitivity of detection of interactions between array elements and sample constituents. Therefore, there is a need for innovative approaches that can increase the number of molecular species in an array without reducing the size of individual elements.

Currently, there are two different technologies established to make microarrays—in situ synthesis method; and Deposition of pre-synthesized DNA.

The two methods differ in the length of the probes deposited. In situ synthesis methods typically use small-length probes due to complexity of individual synthesis steps. For example, the Affymetrix microarrays usually consist of 20-mer probes. The deposition of presynthesized DNA can involve longer probes, even complete cDNAs (complementary DNAs that are made from reverse transcription of the messenger RNAs present in the cell). Alternatively, the Polymerase Chain Reaction products can be used as probes. The limitations of current technologies include high cost of manufacture, low resolution and sensitivity, lack of customization, low array density, and requirement of specialized and expensive instrumentation.

A method for fabricating microarrays of biological samples has been described (see Brown et. al., U.S. Pat. No. 5,807,522). The method involves dispensing a known volume of reagent at each selected array position, by tapping a capillary dispenser on the support under conditions effective to draw a defined volume of liquid onto support. The method can be used to dispense distinct nucleic acids in discrete spots and therefore, to create microarrays of about 100 or about 1000 spots per 1 square centimeters. Each spot is created by dispensing a volume of liquid between 0.002 and 0.25 nl.

Heyneker (U.S. Pat. No. 6,067,100) teach another method for fabricating arrays of oligonucleotides comprising a solid substrate comprising a plurality of different oligonucleotide pools, each oligonucleotide pool arranged in a distinct linear row to form an immobilized oligonucleotide stripe, wherein the length of each stripe is greater than its width. The oligonucleotides are attached to the solid matrix covalently. Alternatively, each oligonucleotide species is attached to fibers individually and then assembled into a strip on a solid support. Such strips from multiple oligonucleotide pools can be arranged side to side on a solid support to obtain a composite array. The presence of a solid support backing, which preferably is plastic, is always necessary and the use of these arrays in the absence of a solid support is not contemplated.

Walt et al (U.S. Pat. No. 5,244,636) describe a fiber optic sensor which is able to conduct multiple assays and analysis concurrently using molecules immobilized at individual spatial positions on the surface of one of the ends of the optical fiber bundle. The fiber optic bundle can be used to transmit excitation light of suitable wavelength to the molecules at the optical fiber end and also for transmission of the emission light back for detection. An array of oligonucleotides or peptides or any other molecules can be created on the ends of optical fibers and used as a microarray.

Multiple uses of microarrays have been described. One of the primary applications is determination of the nucleic acid or protein composition of a sample. Fodor et al (U.S. Pat. No. 5,800,992) detail a method to compare the composition of two or more samples by labeling members of each of the samples with a distinct labeling molecule, preferably fluorescent molecules. The microarrays described by Fodor et al have at least 1,000 distinct polynucleotides per cm².

A use of protein microarrays has been described by MacBeath et. al. Miniaturized assays were developed that accommodate extremely low sample volumes and enable the rapid, simultaneous processing of thousands of proteins. A high-precision robot was used to spot proteins onto chemically derivatized glass slides at high spatial densities. The proteins attached covalently to the slide surface yet retained their ability to interact specifically with other proteins, or with small molecules, in solution. Three applications for the protein microarrays thus generated were described: screening for protein-protein interactions, identifying the substrates of protein kinases, and identifying the protein targets of small molecules.

Another revolutionary technology with implications for biological sciences is the microfluidic chip technology. Microfluidic assays promise to enhance the throughput of biochemical and pharmaceutical analysis. Typically, microfluidic assays are conducted in glass or plastic devices with channels in the order of 10-1000 micron width and height. The reagents for the assays are added to the channels and allowed to react. The output of the reaction is measured by a detectable change in the reactants.

One of the limitations of microfluidic assays is difficulty in concentrating a reactant or separating the product from the reactant. This is important when the assay being used is a multistep process with the products produced in one step being used for reactions in the next step. To achieve this goal, solid phase components are used in microfluidic devices. Most common is the use of micro-particles such as beads, which have been functionalized with a specific affinity for the desired or undesired products. By holding the beads stationary while moving the fluids separation or concentration of the captured product can be achieved. However, the handling of the beads in microfluidic devices is very difficult and usually results in clogging. It also limits the use of microfluidic devices to one assay without extensive cleaning. These limitations have prevented the development of a robust microfluidic system for biochemical analysis.

It is, therefore, an object of the present invention to provide improved fluidic methods and devices for analysis of samples using molecular arrays.

### BRIEF SUMMARY OF THE INVENTION

The present invention describes a novel approach to perform fluidic assays in which the immobilization of the products is carried on substrates that are not part of the fluidic chip itself. The substrates carrying the desired molecular array are inserted into a fluidic chip to generate a fluidic array device for performing sample analysis. The detection of the results of the sample analysis can be performed by an analysis of the substrate while the substrate is still enclosed in the fluidic chip or after the substrate is removed from the fluidic chip. Additionally, if the substrate used to create the molecular array is an optical fiber, the substrate can also provide a conduit for introduction of excitation light for fluorescence analysis of the array.

In general the invention involves molecular arrays on substrates that can be inserted into a fluidic device when needed. Thus, the fabrication of the chip is separated from the fabrication of the array. When used in combination with a sample that is introduced into the fluidic chip, the molecular array on the substrate is allowed to come into contact with the sample constituents. Subsequently, any interaction between the sample constituents and the molecular array can be detected.

It is yet another object of the present invention to describe methods for post-fabrication customization of fluidic chips.

It is another object of the present invention to provide methods to make such arrays and fluidic chips.

It is yet another object of the present invention to describe the use of these arrays without using a fluidic device. One of the advantages using arrays of the present invention in this embodiment is that the targets in the analysis solution are freely mobile across the substrate, and will result in higher kinetic rate of reactions.

### BRIEF DESCRIPTION OF THE SEVERAL VIEWS OF THE DRAWING

FIG. 1 is a view of one embodiment of the invention in which the array elements are distributed on a rod substrate.

FIG. 2 shows the alternative configurations of the invention in cross-sectional view.

FIG. 2A shows the cross-sectional views of various substrates that can be used for fabricating the invention embodiment.

FIG. 2B shows the cross-sectional distribution of material used to create the array elements on the substrate.

FIG. 3 shows how the invention can be used in microfluidic assays.

FIG. 3A shows the configuration in which an array of the invention is introduced into and/or protrudes out of a fluidic channel on a chip from the fluid inlet port.

FIG. 3B shows an alternative configuration in which an array of the invention is introduced into and/or protrudes out of a fluidic channel on a chip from the fluid outlet port.

FIG. 3C shows yet another configuration in which an array of the invention protrudes out of both inlet and outlet ports.

FIG. 3D shows a preferred embodiment in which an array of the invention protrudes from an opening, which is not being used as a fluid inlet or outlet port.

FIG. 4 shows another embodiment of the invention in which multiple substrates containing array elements are joined together to create a larger two-dimensional array.

FIG. 4A shows 10 arrays arranged parallel to each other.

FIG. 4B shows the top view of the larger two-dimensional array created by joining the 10 arrays together using edge pieces on one end of the arrays.

FIG. 4C shows the side view of the larger array to show the edge pieces used to assemble the arrays.

FIG. 5 shows the use of a larger two-dimensional array in conjunction with a fluidic chip.

FIG. 5A shows a two-dimensional array of the invention being used with a 4-channel chip, in which each channel contains a separate fluid inlet and a separate fluid outlet.

FIG. 5B shows a two-dimensional array of the invention being used with a 4-channel chip, in which all four channels are connected and have a single fluid inlet and a single fluid outlet.

FIG. 6 shows a detection approach in which emission light for fluorescence detection in launched into the arrays of the invention from the end of the array and the excitation signal is captured from the top or the bottom of the array.

FIG. 7 shows an alternative method to use the arrays of the invention.

### DETAILED DESCRIPTION OF THE INVENTION

Before providing a detailed description of the inventions of this patent, particular terms used in the patent will be defined.

An “array” is a device comprising a substrate that contains on its surface distinct spots or deposits of one or more than one molecular species. An example of an array in common use is the DNA microarray.

An “element” of an array is a distinct spot or deposition of molecules in a spatially localized area on the substrate of the array.

“Hybridization” is the process by which two strands of DNA or RNA come together to form a double-stranded molecule. For hybridization between two strands to take place, the sequence of the two strands must be completely or nearly so complementary.

“Complementary” strand of a given strand is a strand of DNA or RNA that is able to hybridize to the given strand and is characterized by the presence of nucleotides A, C, G, and T, respectively opposite to nucleotides T, G, C, and A, respectively, on the given strand.

A “Fluidic Chip” is a device comprising a substrate that contains at least one channel and at least one opening that connects the channel to the outside.

A “Fluidic Array Device” is a device comprising a fluidic chip, as defined above, and an array, as defined above.

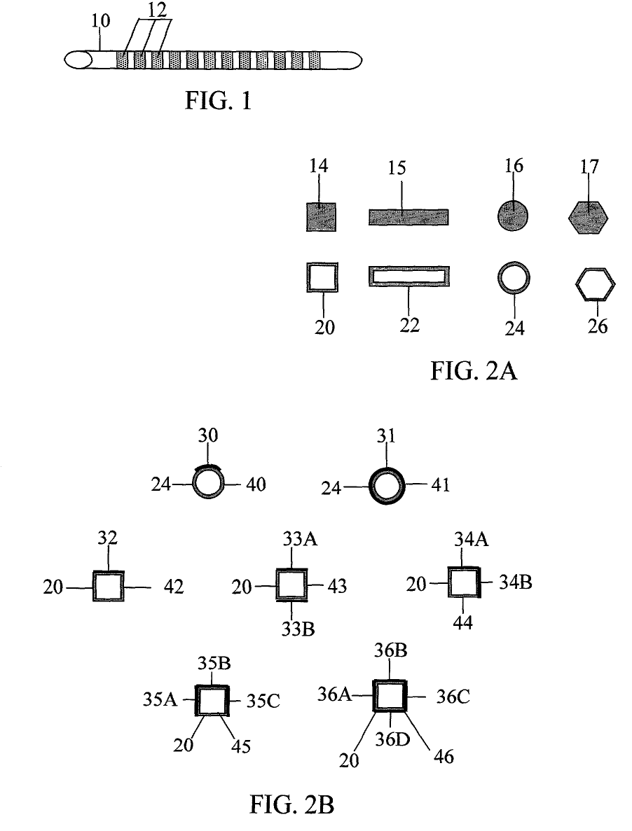

The arrays of the present invention are described with reference to FIG. 1. The array of the present invention 10 comprises a substrate that contains one or more molecular deposition elements 12 on defined segments of the substrate. The array thus generated is a spatially defined array or an addressed array in which the position of each element 12 is predetermined. In another embodiment of the present invention, the elements 12 consist of depositions of samples whose composition or identity of constituents is completely or partially unknown. The elements 12 consist of DNA, RNA, protein or any other chemical or biological species or multiple species. The substrate used to fabricate the array can be transparent, translucent or opaque. However, a transparent substrate is preferable in order to allow optical detection.

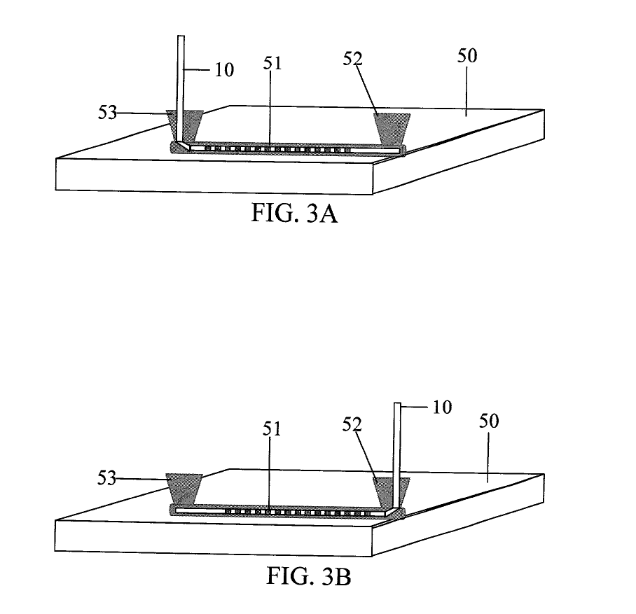

FIG. 2 shows different embodiments of the array of the present invention 10. FIG. 2A shows examples of substrate cross-sections; it will be obvious to anyone that other substrate configurations are equally suitable for this approach. The substrate used can have either a solid core or a hollow core. Examples of solid core substrates shown in FIG. 2A include a square cross-section substrate 14, a rectangular cross-section substrate 15, a circular cross-section substrate 16, and a hexagonal cross-section substrate 17. Examples of hollow core substrates shown in FIG. 2A include a square cross-section substrate 20, a rectangular cross-section substrate 22, a circular cross-section substrate 24, and a hexagonal cross-section substrate 26. FIG. 2B shows examples of the cross-sectional distribution of deposited material used to create the array elements on the substrate. The array 40 comprises a substrate 24 and a material deposition 30 that does not cover the whole circumference of the substrate cross-section. The array 41 comprises a substrate 24 and a material deposition 31 that covers the whole circumference of the substrate cross-section. The array 42 comprises substrate 20 and material deposition 32 that covers only one side of the square substrate cross-section. The array 43 comprises substrate 20 and material depositions 33A and 33B that cover the two opposing sides of the square substrate cross-section. The material depositions 33A and 33B could comprise identical or different materials. The array 44 comprises substrate 20 and material depositions 34A, and 34B that cover the two adjacent sides of the square substrate cross-section. The material depositions 34A and 34B could comprise identical or different materials. The array 45 comprises substrate 20 and material depositions 35A, 35B and 35C that cover three sides of the square substrate cross-section. The material depositions 35A, 35B and 35C could comprise identical or different materials. The array 46 comprises substrate 20 and material depositions 36A, 36B, 36C and 36D that cover each of the four sides of the square substrate cross-section. The material depositions 36A, 36B, 36C and 36D could comprise identical or different materials. It will be obvious to anyone skilled in the art that when substrates with other cross-sections are used, the above principles of circumferential coating or partial circumference coating or coating with different material depositions can be employed.

The cross-sectional dimensions of the substrates will be between 1 micrometer and 10 centimeters, preferably between 10 micrometer and 10 millimeters. The length of the substrates is between 100 microns and 10 centimeter, preferably between 1 centimeter and 5 centimeter. The size of the elements on the substrate is between 10 micrometers and 1 millimeter. The shape of the elements on the substrate could be round, square, oval, irregular or any other shape.

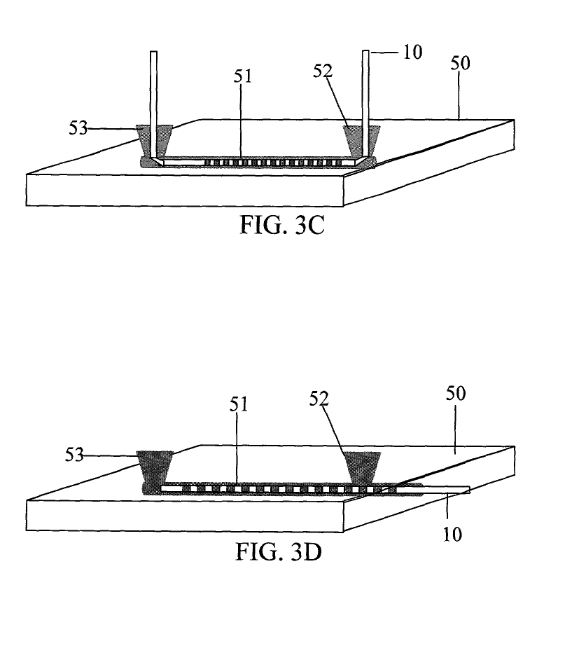

Representative examples of the array 10 in use are shown in FIG. 3. For use, the array 10 of the invention is introduced into a fluidic channel, typically in a fluidic chip. FIG. 3A shows the configuration comprising an array 10 of the invention introduced into the fluidic chip thorough a port that is also used as a fluid inlet for that channel. FIG. 3B shows an alternate configuration comprising an array 10 of the invention introduced into the fluidic chip thorough a port that is also used as a fluid outlet for that channel. FIG. 3C shows yet another configuration comprising an array 10 of the invention introduced into the fluidic chip such that it traverses the channel and its ends are protruding thorough both the inlet and outlet ports for that channel. FIG. 3D shows yet another configuration comprising the array 10 of the invention protruding from a port that is not used for fluid inlet or outlet. In all of these configurations, the array 10 of the invention can be inserted or removed after the fluidic chip has been assembled without having to dismantle the chip. As will become obvious in further discussion, the configuration shown in FIG. 3D is preferable for the ease of introduction of arrays into fluidic chips. In yet another configuration, the array 10 of the invention can be inserted into a fluidic channel such that it does not protrude out of the channel. In this configuration, the array cannot be removed after insertion, but still allows post-fabrication customization of the chips.

A number of methods can be used to fabricate the arrays 10 of the present invention. To generate an array 40 or array 42, both shown in FIG. 2B, or any other similar array, the substrate can be held flat and the material deposited by either a liquid dispensing system e.g. inkjet printing head, or a pen that is used to draw a line on the substrate.

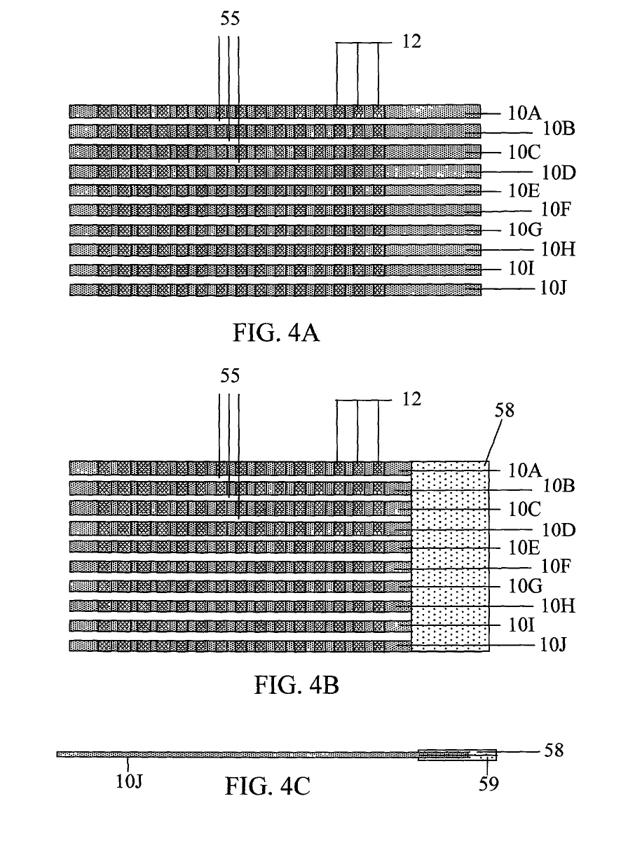

Since most fluidic devices contain more than one channel, a preferred embodiment of the invention will be arrays that can be inserted into multiple channels simultaneously. A method to assemble such two-dimensional arrays is shown in FIG. 4A, 4B and 4C that comprises of three steps: 1) fabricate multiple arrays 10 (shown by 10A, 10B . . . , 10J) consisting of different or similar array elements 12 on each array; 2) arrange them parallel to each other leaving a gap 55 between each adjacent pair of arrays; 3) attach them together on one end using solid substrates 58 and 59 while maintaining them in a parallel configuration. There is no need of a backing matrix. It will be immediately obvious to anyone skilled in the art that the array described in FIG. 4C can be stacked atop each other to create a three-dimensional array that still maintains its ability to be introduced into a fluidic device.

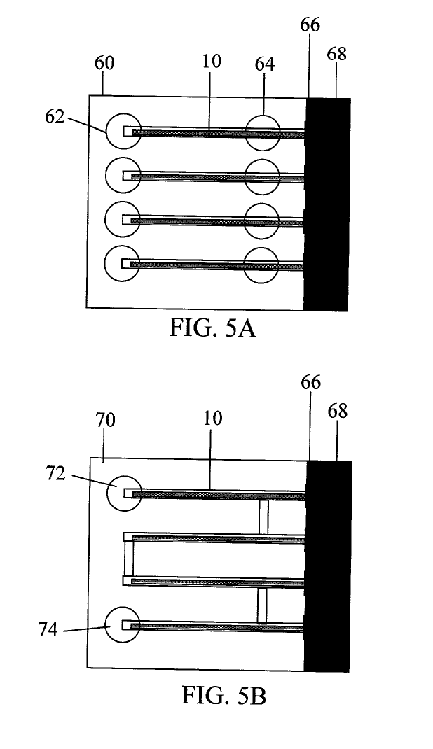

FIG. 5A and FIG. 5B shows the method of using a two-dimensional assembly of the arrays in conjunction with fluidic chips. FIG. 5A shows a 4-channel fluidic chip with a two-dimensional array comprising four arrays 10 of the invention, in which each channel contains a separate fluid inlet and a separate fluid outlet. The sample in each channel comes in contact with one array of the two dimensional assembly. One particular application of such configuration will be in processes in which a large number of samples need to be tested against a set of molecular array elements.

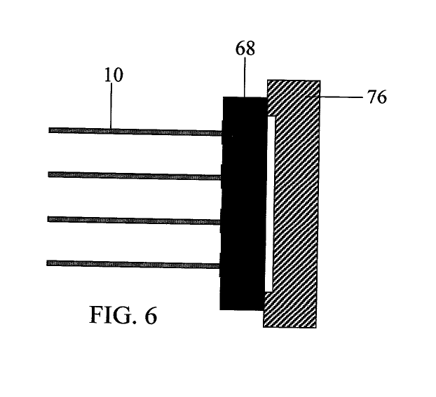

FIG. 6 shows a detection method using a light source 76 coupled to a two-dimensional assembly comprising four arrays 10 of the invention. The substrate used to create the array of the invention is a material that can transmit light of suitable wavelengths and is therefore, an optically transparent material for those wavelengths, e.g. glass and optically clear plastics. The solid substrate 68 used to create the assembly of the arrays 10 of the invention is optically opaque. The light source 76 can be a line source with a line width of 1 mm and line length corresponding to the length of substrate 76. The arrangement of the light source and the two-dimensional assembly is such that light from source is launched into the arrays 10. The detection of any fluorescent material present on the arrays 10 can be detected with a suitable optics.

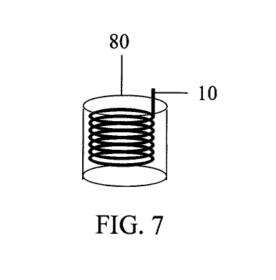

In addition to inserting the arrays into fluidic devices for exposure to samples, the arrays can also be used with other fluid-holding containers. FIG. 7 shows how an array 10 of the invention can be used in combination with a well 80 of a microtiter plate. The array 10 is rolled up into a spiral with a diameter less than that of the microtiter well 80. After exposure to the sample, the array 10 can be removed from the well 80 and analyzed.

In another embodiment, molecular depositions are made on a thin substrate e.g. 150-micron glass or plastic. The substrate material in between the molecular depositions is removed to one edge of the substrate, leaving the areas of molecular depositions held together by the other edge of the substrate. Such removal of the substrate can occur either before or after the depositions. Glass sheets in the thickness of 50 micrometer are commercially available and can be used for this purpose. Alternatively, plastic sheets with thickness as little as 10 microns or less can be used. To increase the firmness of plastic substrate, it can be supported with glass or metal inserts.

One of the advantages of these arrays is that the target molecules are able to diffuse faster between different locations and reach the corresponding probe. Another advantage of the present arrays is that amount of surface area available for spotting is larger than conventional arrays and therefore, a larger number of probes can be exposed to the targets in the sample simultaneously.

The linear depositions of functionalization can be made on the substrate using any of a number of methods. The functionalization can be performed by drawing using rollers, pens or quills or by printing using inkjet or bubble jet printers. Additionally for polymeric biological molecules such as DNA, proteins and RNA, the appropriate functionalization can be added to the fiber using in situ synthesis using photolithography or ink jet printing.

The molecules that are deposited on the substrates are usually covalently coupled to the substrate material. The choice of a particular method for coupling specific molecules to a substrate depends on characteristics of the molecules and the substrate. For example, a number of methods are known in the art for coupling DNA molecules to glass substrates, including coupling of amino-terminated nucleotides to aldehyde coated glass substrates. Similarly, a number of methods for coupling protein molecules to plastic substrates are known in the art, and can be used to create the arrays of the present invention.

In another embodiment, the elements of the array are created on both surfaces of a substrate. The arrays on the two surfaces of a substrate can consist of the identical spots or different spots. If the array on the two surfaces consist of identical spots, they can be detected simultaneously or separately. The advantage of simultaneous detection is higher sensitivity; the advantage of having different spots and separate detection is increase in density of elements of the array.

The detection of products captured on the elements of the array can be done by a number of detection techniques. The products captured on the elements can be studied in situ with fluorescence or by selective release from the fiber. Or the arrays can be removed from the device and then analyzed by fluorescence or other biophysical techniques such as mass spectrometry after release of the product.

One particular use of the arrays of invention is analysis of DNA or RNA samples by hybridization. Another use is to study interaction of proteins with DNA or with other proteins or small molecules e.g. antibody-antigen interactions.

The deposition of the molecules on the substrate can be performed by drawing using rollers, pens or quills. Additionally for polymeric biological molecules such as DNA, proteins and RNA, the appropriate deposition can be performed on the substrate using in situ synthesis, e.g. using photolithography or ink jet printing. Multiple fibers can be laid parallel to each other for the deposition process.

Any chemistry that has been described in microfluidics and uses beads can be modified to work with fibers. Examples of such technologies include Genetic Bit Analysis, scintillation proximity assay, etc.

The arrays of the invention can also be combined with molecular biology reagents and instructions to design kits for genomic and proteomic research as well as for drug discovery.

Although the invention has been described in some detail by way of illustration and example for purposes of clarity and understanding, it may be readily apparent to those of ordinary skill in the art in light of the teachings of this invention that certain changes and modifications may be made without departing from the spirit or scope of the appended claims.

### EXAMPLE 1

### Fabrication of an Array on a Substrate

Take a square cross-section borosilicate glass tube with each side measuring 330 microns and use them for creating the substrate. Attach amino functional groups to the surface of the substrate by treating it with N-(2-Aminoethyl)-3-aminopropyltrimethoxysilane. Spot human cDNA molecules of interest on the substrate using a felt-tip pen. Allow the cDNA molecules to attach to the amino groups and wash. Dry the substrates. The arrays are now ready for use.

### EXAMPLE 2

### Creating a Two-dimensional Assembly of Arrays

Take four square cross-section borosilicate glass tube, 20 mm long, with each side measuring 330 microns and treat them for attaching the amino functional group as in example 1. Place them parallel to each other in a fixture at a spacing of 330 microns. Make sure that the substrates extend 5 mm beyond the fixture at one of their ends. Using a felt tip pen, draw lines across the substrates. Take two pieces of polycarbonate, 10 mm square, to use as edge pieces. Machine four grooves in each of them at a spacing of 330 microns, each groove measuring 330 microns wide and 165 micron deep. Align the ends of the four arrays extending beyond the fixture with the four grooves in the edge pieces and bond the edge pieces together, holding the arrays together.

### EXAMPLE 3

### Fabrication of a Fluidic Chip

Fluidic chips will be made from two pieces of polycarbonate. Take a square piece of polycarbonate sheet, 2 mm thick, and 20 mm on each side and use it as the chip base. Machine four grooves in the chip base 400 microns wide and 400 microns deep such that they extend from one edge to 4 mm away from the other edge. These grooves will serve as channels. Take another piece of polycarbonate with similar dimensions and use it as the chip top. In the chip top, drill eight holes to correspond to four channels on the chip base, each channel, therefore, having a fluid inlet and fluid outlet through the chip top. Assemble chip top and chip base, carefully aligning the channels in the chip base and holes in the chip top. Join the chip top and chip base using acetone. In this assembled chip, in addition to having a fluid inlet and fluid outlet, each channel also has a port on the side, which can be used for introduction of the array 10 of the invention.

### EXAMPLE 4

### Analysis of a DNA Sample

Make a human cDNA array as described in example 1. Make a fluidic chip as described in example 3. In order to create fluidic array device able to perform human cDNA array analysis, insert the human cDNA array into one of the channels of the fluidic chip. Take a DNA sample of interest and label the DNA molecules present in the sample with Cy3. Add the fluorescently labeled sample and introduce it into the fluidic array device. Let the target molecules in the sample hybridize to the probes for 1 hour. Take the array out and wash with 0.1 mM TE buffer (10 mM Tris HCl, 0.5 mM EDTA). Position the array under a fluorescent microscope equipped with a digital camera. Use an excitation light of 550 nm wavelength and observe and record the light intensity from each element at 570 nm emission wavelength. If the sample contains targets that complementary to the probes on the array, the light intensity recorded from the corresponding element(s) will be stronger than others.

What I claim as my invention is:

1. Any fluidic array device comprising an assembly of a fluidic chip and a substrate containing a pre-defined molecular array, the said fluidic chip and the said substrate having been fabricated separately.

2. A method for analysis of a sample involving use of a device of claim 1.

3. A device of claim 1, comprising the said substrate contains a molecular array of polynucleotides.

4. A device of claim 1, comprising the said substrate contains a molecular array is an array of polypeptides.

5. Any fluidic chip that is assembled with a substrate containing a pre-defined molecular array, to fabricate a fluidic device of claim 1.

6. Any substrate containing a pre-defined molecular array that is assembled with a fluidic chip, to fabricate a fluidic device of claim 1.

7. Any method of sample analysis comprising the insertion a substrate containing a predefined molecular array in to a fluidic chip to create a fluidic device of claim 1 for further processing or analysis of molecular array.

8. Any method of sample analysis comprising the removal of a substrate containing a predefined molecular array from a fluidic device of claim 1 for further processing or analysis of molecular array.

9. A molecular array of claim 6 comprising array elements are circumferentially around the substrate.

10. A molecular array of claim 6 comprising more than one substrate such that the array elements are distributed in two space dimensions.

11. A device of claim 1 comprising an assembly of a fluidic chip and a substrate containing a pre-defined molecular array where the array substrate is not fully enclosed in the fluidic chip.

12. A device of claim 1 comprising an assembly of a fluidic chip and a substrate containing a pre-defined molecular array where the array substrate is fully enclosed in the fluidic chip.

13. An array of claim 6 comprising a light source to introduce excitation light for detecting the level of fluorescence on the array elements.

14. An array of claim 10 comprising one or more light sources to introduce excitation light for detecting the level of fluorescence on the array elements.

15. Any molecular array comprising a substrate more than 1 cm. in length and cross-sectional area of less than 1 mm², the said substrate containing a pre-defined depositions of molecules on its surface along its length.

16. A molecular array of claim 15, comprising the depositions of molecules are circumferential.

17. A molecular array of claim 15, comprising the depositions of molecules on the surface of the substrate consist of different species of molecules on different aspects of the cross-section.

18. A molecular array of claim 15, comprising the substrate is an optically transparent material and is used for transmitting excitation light to the array elements.

19. A method for the use of a molecular array of claim 15, comprising the array is used in combination with a microtiter plate.

20. A two-dimensional array of claim 10 comprising the elements deposited on each constituent substrate are different.
